(19) United States
(12) Patent Application Publication
(10) Pub. No.: US 2005/0148505 A1
(43) Pub. Date: Jul. 7, 2005

(54) Genes and agents to regulate follicular development, ovulation cycle and steriodogenesis

(76) Inventors: Tae H. Ji, Lexington, KY (US); Inhae Ji, Lexington, KY (US)

Correspondence Address: MCDERMOTT WILL & EMERY LLP, 600 13TH STREET, N.W., WASHINGTON, DC, 20005-3096, US

(21) Appl. No.: 10/736,892

(22) Filed: Dec. 17, 2003

Related U.S. Application Data
(60) Provisional application No. 60/437,729, filed on Jan. 3, 2003.

(51) Int. Cl. A61K 38/24; C12Q 1/68; C07H 21/04; C07K 14/575

(52) U.S. Cl. 514/12; 435/6; 536/23.5; 530/399

(57) ABSTRACT

Methods of regulating gene expression through exposure of the gene to follicle stimulating hormone are provided. Follicle stimulating hormone is used to suppress the expression of T3-binding protein mRNA and thereby regulate ovulation, estrogen production and steroidogenesis.

### CROSS REFERENCE TO RELATED APPLICATION

This application claims priority to provisional application U.S. Ser. No. 60/437,729 filed Jan. 3, 2003.

### FIELD OF THE INVENTION

The present invention relates to the use of follicle stimulating hormone (FSH) to regulate gene expression. More particularly, this invention relates to the use of follicle stimulating hormone to suppress the expression of T3-binding protein mRNA.

### BACKGROUND OF THE INVENTION

FSH stimulates granulosa cell differentiation and follicular development. It is responsible for inducing estrogen production and preventing the apoptosis of early antral follicle cells in rodents. In growing follicles, FSH mediates continued mitotic activity of granulosa cells and decreased FSH responsiveness is associated with follicular atresia. These FSH activities are initiated when FSH binds to and activates the FSH receptor. FSH receptor mRNA is expressed in granulosa cells as early as the primary stage of follicular development. The importance of FSH and its receptor is clear as female mice homozygous for a defective FSHβ are infertile due to the arrest of follicular development at the preantral stage. The ovarian phenotype of an FSH receptor knockout mice is similar to that observed in the FSH knockout mice. It has been shown that FSH elicits peptide and steroid hormone production in granulosa cells by inducing the expression of its target genes. Due to the broad scope of the FSH effects, a large number of genes are likely responsive to the hormone. However, only a limited number of FSH-regulated genes have been identified, to date, such as inhibin/activin subunits and steriodogenic enzymes. In particular, little is known about the FSH responsive genes at the preantral stage. This is, in part, due to the lack of a suitable experimental system. Thus, there is a need for methods to identify and characterize genes that are regulated by FSH at the preantral stage.

### SUMMARY OF THE INVENTION

In one aspect of the invention there are provided methods to regulate follicular development, ovulation, steroid hormones production, associated health related disorders and diseases in female and male humans and mammals by modulating the genes and gene products of cytosolic T₃-binding protein, 3alpha-hydroxysteroid dehydrogenase, gene products including their proteins, and thyroid hormone, T3, the newly discovered genes, and their related molecules. In particular, the invention provides a method of modifying cytosolic T₃-binding protein (CTBP) gene expression comprising contacting the cytostolic T₃-binding protein gene with an effective amount of follicle stimulating hormone (FSH).

In another aspect of the invention there is provided a method for modifying preantral stage and/or early antral stage follicular development in a mammal comprising exposing the follicles of the mammal to an effective amount of a compound that activates the adenylyl cyclase/cAMP signal pathway.

In another aspect of the invention there is provided a method of modifying CTBP gene expression in granulosa cells comprising contacting the granulosa cells with an effective amount FSH.

In a further aspect of the invention there is provided a method of enhancing aromatase activity in granulosa cells comprising contacting the granulosa cells with an amount of FSH effective to suppress CTBP gene expression.

In yet another aspect of the invention there is provided a method of modifying estrogen production in a mammal comprising administering to the mammal an effective amount of follicle stimulating hormone.

In a further aspect of the invention there is provided a method of modifying ovulation in a mammal comprising administering to the mammal an effective amount of follicle stimulating hormone.

In another aspect of the invention there is provided an isolated nucleic acid having the sequence of SEQ ID No.13 and nucleic acid molecules that hybridize to SEQ ID NO.13 under high stringency conditions. There is also provided an isolated polypeptide having the amino acid sequence of SEQ ID NO. 12.

In another aspect of the invention there is provided a method for modifying the expression of the gene encoded by SEQ ID NO.13, said method comprising exposing the gene to an effective amount of FSH.

### BRIEF DESCRIPTION OF THE DRAWINGS

FIG. 1. A). Nucleotide sequence alignment of rat CTBP cDNA. The nucleotide sequence of the open reading frame of CTBP cDNA (SEQ ID NO. 7) is aligned with the matching part of the rat μ-crystallin cDNA sequence (SEQ ID NO. 8) (GeneBank accession no. Y17328), showing one base mismatch. B). The rat (SEQ ID NO. 8), mouse (SEQ ID NO. 9) and human (SEQ ID NO. 11) μ-crystallin amino acid sequence are shown.

FIG. 2. Localization of inhibin α and CTBP mRNAs in adult rat ovary. Tandem ovarian sections of an adult rat ovary were hybridized with antisense probes for inhibin α (A) and CTBP (B and C), and subjected to liquid emulsion autoradiography (left panels) followed by hematoxylin staining (right panels). Both inhibin α and CTBP mRNA signals are seen in granulosa cells of preantral follicles (arrow) and early antral follicle (arrowhead), but not in atretic follicles (AtF) and corpus lutea (CL). GC, for granulosa cell; T, theca cell; InT, interstitial cell. Photographs are taken at 20× magnification for A and B, and 200× for C.

FIG. 3. CTBP mRNA expression in primary granulosa cell culture. Granulosa cells were isolated from the ovaries of 24 day old immature rat primed with 17β-estradiol. The cells were cultured in the presence of 30 ng/ml FSH for up to 48 h, and RNA was extracted and amplified for CTBP using quantitative RT-PCR. In addition, L-19 mRNA was probed as an internal control. The CTBP values were normalized with the corresponding values of L-19 and two independent experimental values were averaged.

FIG. 4. Follicular stage dependent expression of CTBP mRNA. Immature 22-23 day old rats were primed with a single injection of PMSG for 0 (A), 3 (B), 6 (C), 24 (D) and 48 (E) h, followed by priming with an hCG injection for 1 (F) and 6 h (G, H). The ovaries were excised, sectioned and hybridized with the CTBP antisense probe (A-G) or inhibin a antisense probe (H). In addition to the dark field autoradiographs in the left panel, the corresponding bright field images of hematoxylin staining are aligned in the right panels. Arrows and arrowheads indicate preantral and early antral follicles, respectively. PO, preovulatory follicle; AtF, atretic follicle. 40× magnification.

FIG. 5. Effects of cycloheximide and α-amanitin treatment on the FSH/forskolin-induced expression of CTBP mRNA. Granulosa cells were isolated from 17β-estradiol primed 24-day old immature rat ovaries and cultured with FSH (30 ng/ml) or forskolin (10 μM, FSK) in the presence or absence of cycloheximide (10 μg/ml, CHX) or α-amanitin (30 μg/ml, AMA) for the 6 h. CHX, a translation inhibitor, and AMA, a transcription inhibitor, were pretreated to the cell cultures one hour before hormone treatment. Total RNA was isolated and analyzed for CTBP and L-19 mRNA by semi-quantitative RT-PCR assay using 23 cycles for CTBP and 20 cycles for L-19. L-19 was used as an internal control. Values shown are the range of two independent experiments along with the mean, indicated by bars.

FIG. 6. Tissue-specific expression of CTBP mRNA in adult rats. The relative mRNA expression level of the CTBP was compared among different tissues by Northern blotting. 20 μg of total RNA from adult tissues and 4 μg of total RNA from immature rat ovaries were separated on 1.2% agarose gel, hybridized with CTBP probe. The blot was stripped and re-hybridized with L-19 probe. Note strong intensities of CTBP transcript in liver, kidney, brain and immature ovaries, no signal from stomach, pancreas, lung and bladder.

FIG. 7. Effects of T₃ hormone on estrogen production. (A) Granulosa cells were treated with increasing concentrations of FSH and the culture media were assayed for estrogen production. (B) Granulosa cells that were pre-treated with FSH were treated with T3 for varying time periods, 0-4 days. The culture media were assayed for estrogen. (C) Granulosa cells were treated with none, thyroid hormone T3, FSH, or FSH and T3 combo for 3 or 5 days. The culture media were assayed for estrogen production. (D) Granulosa cells were treated with none, thyroid hormone T3, hCG, or hCG and T3 combo for 3 or 5 days. The culture media were assayed for estrogen production.

FIG. 8. Effects of CTBP on suppression of estrogen production by T3. Granulosa cells were transfected with the expression vector carrying the CTBP cDNA, treated with FSH or FSH and T3. They were assayed for estrogen production. In addition, Untransfected granulosa cells were treated with FSH or FSH and T3.

FIG. 9. A). A novel RNAse PH like gene (SEQ ID NO. 11). Clone 30 identified from the differential display shows an open reading frame of 276 amino acids. The sequence shares some homology with bacterial RNAse PH. Since this clone is up-regulated by FSH, it is possible that CTBP mRNA might be degraded by this putative RNAse. Therefore, the putative enzyme could be useful for regulation of steroid production. In addition to clone 30, we have identified another novel gene (clone 22) from the differential display. The structure and function of this novel gene is unclear. 9B). The amino acid sequence encoded by the DNA of SEQ ID NO. 11 (SEQ ID NO. 12). C). The open reading frame of the sequence of 9A (SEQ ID NO. 13).

### DETAILED DESCRIPTION OF THE INVENTION

In search for early genes responsive to FSH, we examined differences in gene expression caused by exposure of rat granulosa cells to FSH using mRNA differential display methodology. Here, we present the evidence that FSH down-regulates expression of nicotinamide adenine dinucleotide phosphate (NADPH)-dependent cytosolic T₃-binding protein (CTBP) in granulosa cells. CTBP appears to play a significant role in the regulation of steroidogenesis and follicular development in the mammalian ovary.

The rat ovarian granulosa (ROG) cell line is a useful system. It was established from immature granulosa cells of the rat ovary and grows in a defined serum-free medium containing activin A, but not FSH. ROG cells show many characteristics of undifferentiated immature cells, lacking steroidogenesis and the LH receptor. Upon exposure to FSH, the cells become post mitotic and highly steriodogenic, similar to mature granulosa cells of a dominant follicle. FSH-stimulated ROG cells also become dependant on the continued presence of FSH and will undergo apoptosis upon its removal. In addition, ROG cells form a structure resembling a follicle when cultured in the presence of an oocyte/cumulus cell complex. The present inventors have previously shown that the actin cytoskeleton in ROG cells quickly rearranges within three hours of exposure to FSH, leading to changes in cell-cell interactions.

FSH plays crucial roles in differentiation of granulosa cells and development of follicles. Considering the broad scope of the FSH effects, a large number of genes are likely responsive to the hormone. However, only a limited number of genes have been identified as FSH-regulated genes, particularly during the preantral stage. In an attempt to better define genes involved in follicular development, we examined primary granulosa cell cultures, an undifferentiated rat ovarian granulosa cell line and rat ovaries, using differential display, quantitative RT-PCR, Northern blot and in situ hybridization. We report, for the first time, that nicotinamide adenine dinucleotide phosphate (NADPH)-dependent cytosolic T₃-binding protein mRNA is expressed in the ovary, particularly in the granulosa cell layer of preantral and early antral follicles, but not in large preovulatory follicles. Its expression markedly declines in response to FSH, which is dependent on the period of the exposure. This FSH-responsive down regulation is dependent on granulosa cell differentiation and follicular development. FSH down-regulates the mRNA via the adenylyl cyclase/cAMP/protein kinase A pathway and the down-regulation requires de novo synthesis of a regulatory protein(s). The cytosolic T₃-binding protein may play a significant role in the regulation of steroidogenesis and follicular development in the mammalian ovary.

Differential Display of mRNA Shows Decrease of CTBP mRNA Expression in Response to FSH

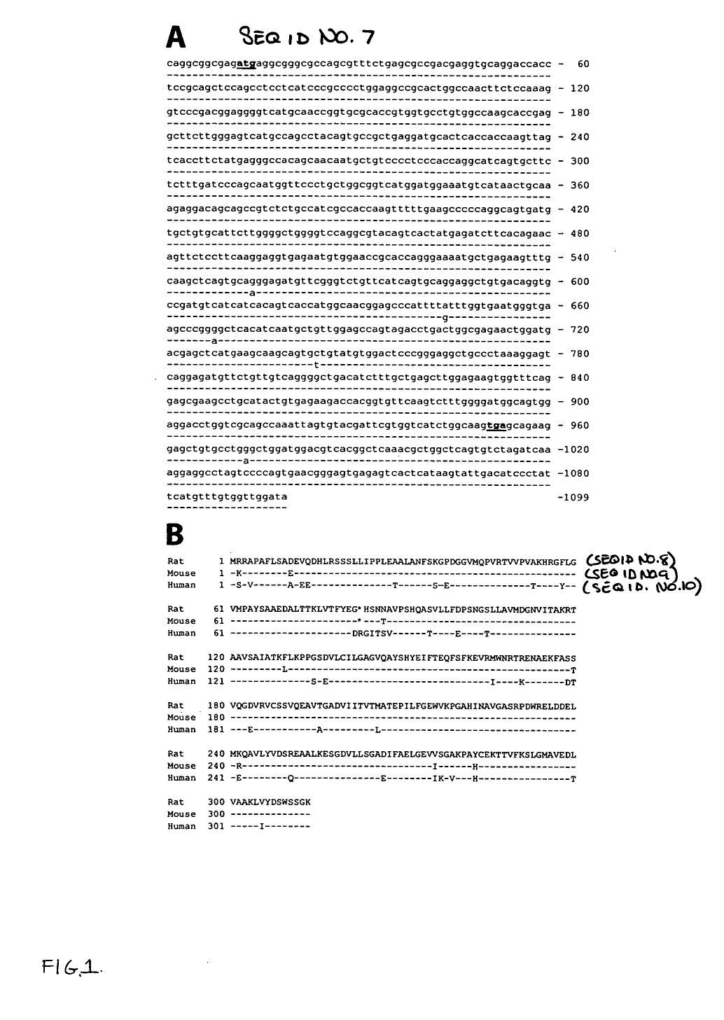

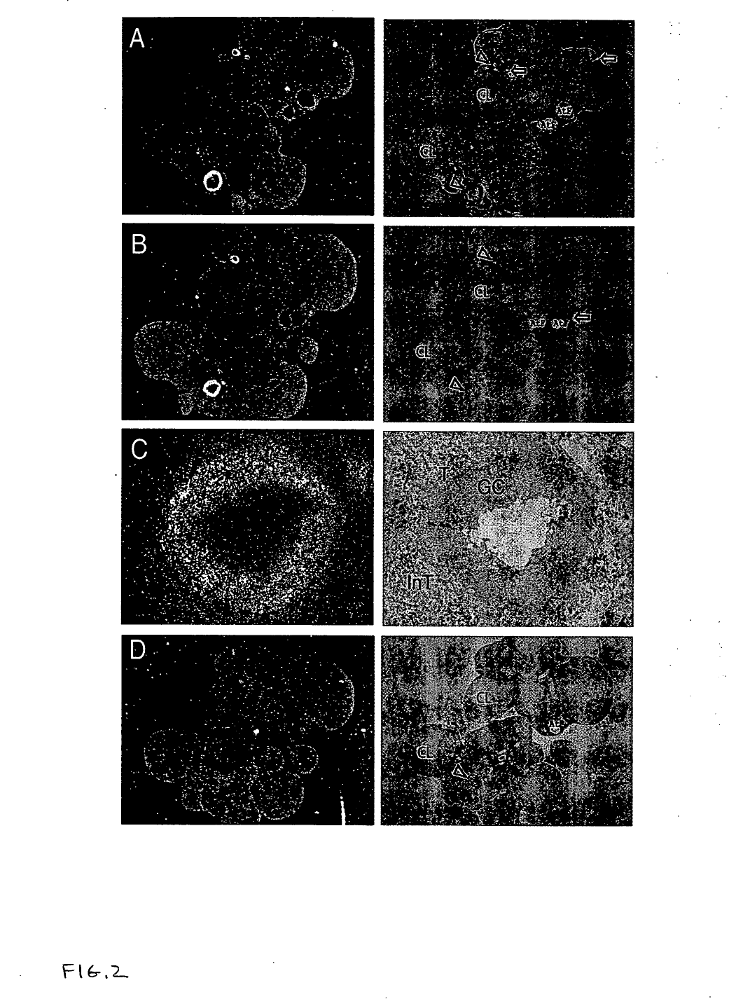

To identify FSH-responsive genes in granulosa cells, ROG cells were cultured in the absence or presence of 30 ng/ml FSH for 6 hours, total RNA was isolated, and mRNA was differentially displayed on sequencing gels (FIG. 1). Bands, which showed intensity difference between control and FSH treated groups were excised, PCR-amplified, cloned and sequenced. One of the clones matched to nucleotides 768-1210 of the full length cDNA of rat μ-crystallin (GeneBank accession no. Y17328), with one base mismatch (FIG. 2) that did not alter the open reading frame or amino acid sequence. Inhibin α gene was also found among the clones.

Crystalline proteins were initially isolated from the transparent eye lens and therefore their distribution had been thought to be restricted to the lens and have only refractive functions. However, they share no significant nucleotide and amino acid sequence homology, and are found in tissues other than the eye lens, suggesting other functions. In fact, based on the amino acid sequence homology with enzymes, several non-lens functions have been suggested for μ-crystallin, such as lysine and ornithine cyclodeaminase and a reductase possibly involved in amino acid metabolism. The only demonstrated non-lens function for μ-crystallin is NADP-regulated thyroid hormone binding. In search of thyroid hormone binding protein, a protein was isolated that showed high specific binding affinity to thyroid hormone (T₃) in a NADP-dependent manner. Subsequent amino acid sequencing and cDNA cloning revealed that the protein was identical to μ-crystallin. Recently, another group has clearly demonstrated that the protein binds T₃ and transfers the hormone into the nucleus, where it interacts with its nuclear receptor, the thyroid hormone receptor. Consistent with this, CTBP was also found in thyroid hormone target tissues such as brain, retina, muscle, skin, kidney and liver. Our Northern blot analysis showed similar tissue specific expression.

The rat μ-crystallin is 313 amino acids long, and shares 97%, 87% and 82% amino acid sequence identity with the mouse (GeneBank accession no. AF039391), human (GeneBank accession no. U85772) and kangaroo (GeneBank accession no. M90841) μ-crystallin sequences, respectively. μ-crystallin was originally isolated from kangaroo lens, and thus other homologous genes have been named as μ-crystallins. However, except for kangaroo μ-crystallin, all known μ-crystallins were isolated from non-lens tissues. For example, human μ-crystallin was isolated from kidney cells, and mouse was from skin cells and our clone from ROG cells. Several functions unrelated to lens have been suggested for μ-crystallin, however, the only proven non-lens function is nicotinamide adenine dinucleotide phosphate (NADPH)-dependent CTBP (Vie et al., 1997, Mol Endocrinol 11:1728-36; Mori et al., 2002 Endocrinology 143:1538-44). No specific function has been described for the rat μ-crystallin. Thus, we used CTBP to describe the clone instead of μ-crystallin.

CTBP mRNA Expression in Granulosa Cells of Small Follicles

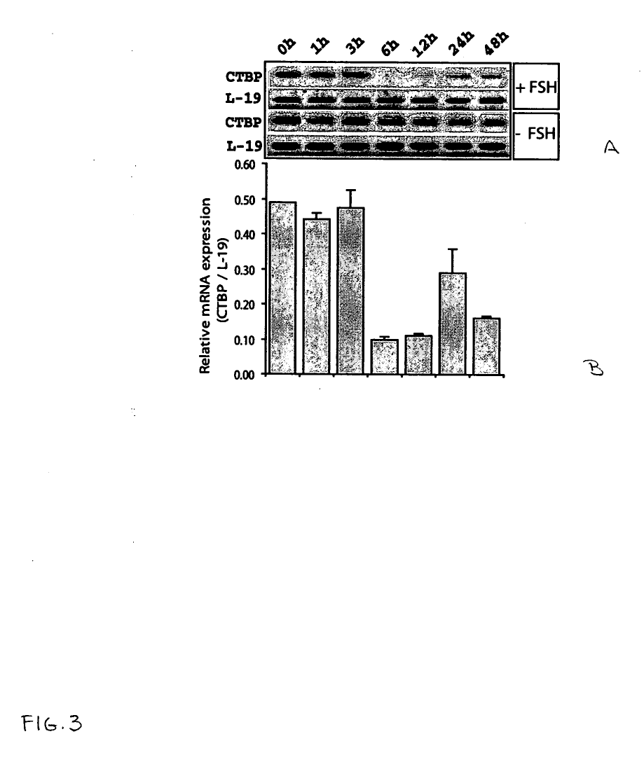

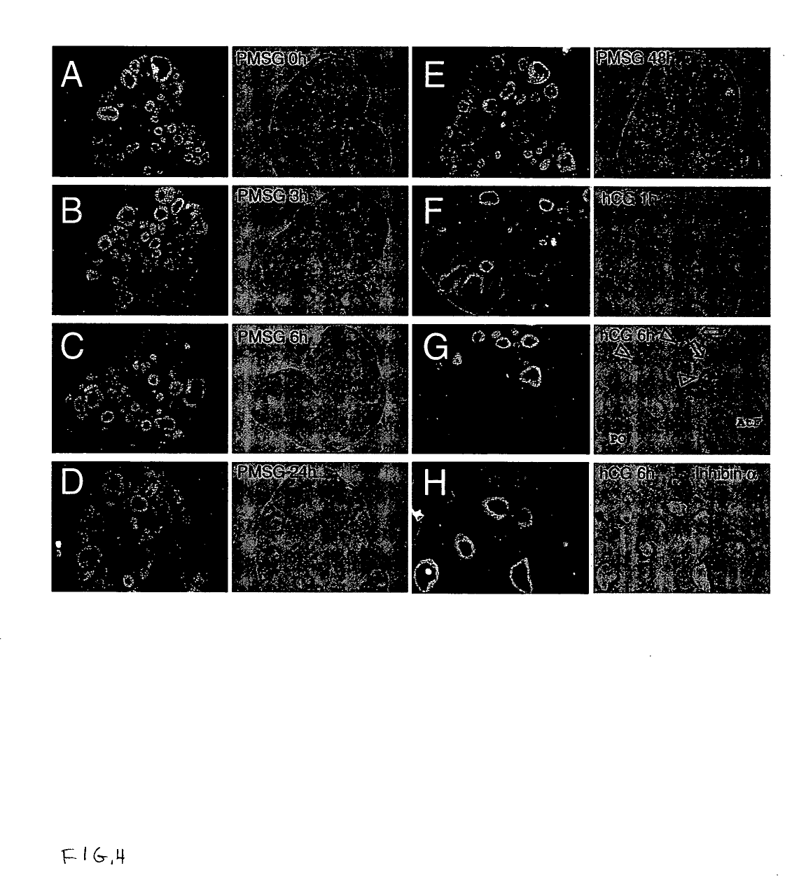

To localize the in vivo CTBP mRNA expression, sections of an adult rat ovary were in situ hybridized. The CTBP mRNA signal was detected only in small, growing follicles but not in atretic follicles and corpus lutea (FIG. 3A). To verify the differentiation and development dependent expression of CTBP, we compared the CTBP expression with that of inhibin α, a known FSH-responsive gene, whose expression is dependent on granulosa cell differentiation and follicular development. Tandem ovarian sections were hybridized with an inhibin α antisense probe. All of the follicles that showed the CTBP mRNA signal expressed inhibin α mRNA, and furthermore, no other follicles showed the signal (FIG. 3A and B). It is important to note that the granulosa cell layers of the small growing follicle were exclusively labeled with the CTBP mRNA probe, when the labeling image in FIG. 3A was magnified (FIG. 3C). No signal was detected from the sections hybridized with CTBP sense probe (data not shown). The result indicates that the CTBP mRNA expression is confined to the granulosa cells in the follicle.

FSH Suppresses CTBP mRNA Expression in Primary Granulosa Cell Culture

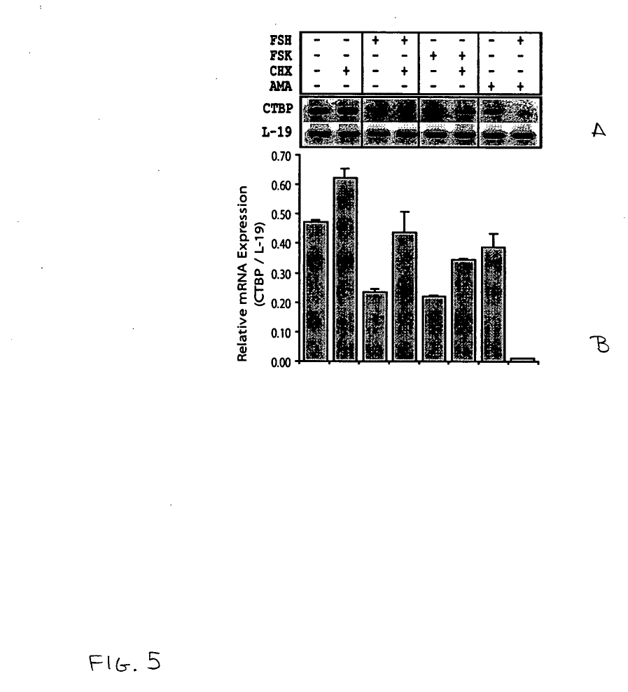

The differential display in FIG. 1 shows that the CTBP mRNA level in ROG cells diminished upon six hours exposure to FSH. Although ROG cells show some of the characteristics of undifferentiated granulosa cells (FIG. 3) as previously reported, we tested the FSH responsiveness of CTBP mRNA in a more physiologically relevant system. Undifferentiated granulosa cells were isolated from immature rats and cultured in the presence or absence of FSH for 1-48 hours. RNA expression level was measured by semi-quantitative RT-PCR. The RT-PCR analyses revealed an intriguing picture of the time- and hormone-dependent expression of CTBP mRNA (FIG. 4). Compared to the abundant expression in untreated granulosa cells, CTBP mRNA was barely detectable within six hours of exposure to FSH. This down regulation lasted until 12 hours, at which time CTBP mRNA levels began to rise. However, even by 48 hours of exposure the mRNA levels were below the original level prior to FSH treatment. In contrast to the dramatic time- and FSH-dependent fluctuation in CTBP mRNA, an internal control, L-19 mRNA, maintained a stable level with marginal variation. The normalized levels of CTBP mRNA support the conclusion of the down- and up-expression of CTBP mRNA in the primary granulosa cell culture during FSH exposure.

Transient Expression of CTBP mRNA Primarily at Early Stage of Follicular Development.

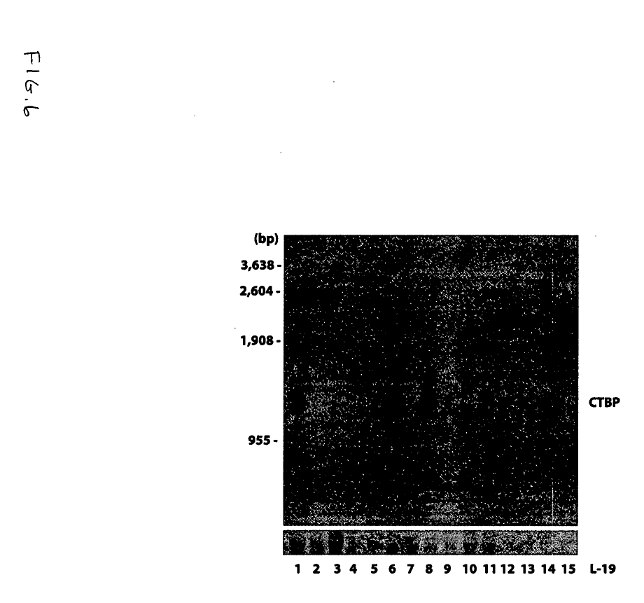

The analysis of CTBP mRNA extracted from the cultured primary granulosa cells, as shown in FIG. 3, reflects the FSH- and time-dependent changes in the cells. However, it is unclear whether those same changes indeed occur in vivo, and if so, whether in the same time-dependent manner and sequence of down- and up-expression. In addition, there is the outstanding question as to whether the down- and up-regulation is also dependent on in vivo cell differentiation and follicle development. In a step to address these questions, in situ hybridization was performed on ovarian sections of immature rats that were primed with PMSG for 0-48 h. In the ovaries of non-primed rats, CTBP mRNA was detected primarily in preantral and early antral follicles (FIG. 5A). Furthermore, it was found exclusively in the granulosa cell layers, consistent with the FIG. 3 result. As expected, PMSG stimulated follicular growth (FIG. 5B-5F). During this follicular development, strong CTBP mRNA signals were observed consistently in small follicles mostly at preantral and early antral stages.

In striking contrast, the signal was weak in large antral follicles (FIG. 5B-5E), which became more obvious as follicles grew. By 48 h exposure to PMSG, many follicles reached the large antral stage and the CTBP mRNA expression was barely noticeable (FIG. 5F). To determine the effect of granulosa cell luteinization, the rats were additionally primed with hCG for 6 h. This hCG treatment completely abolished the CTBP signal in large antral follicles, particularly in preovulatory follicles, whereas a high level of CTBP mRNA expression persisted in small follicles (FIG. 5G). These results indicate that CTBP mRNA is expressed in preantral and early antral follicles, but not in the follicles further developed beyond the early antral stage, particularly in preovulatory follicles. This expression pattern raised the unlikely, yet fundamental question whether the transcription machinery was shut down all together, but not specifically for CTBP transcription, in the large follicles. To test this possibility, a tandem ovarian section was probed for inhibin a transcript. FIG. 5H shows strong signals of inhibin α mRNA in the large antral and preovulatory follicles. This result clearly demonstrates not only that active transcription is ongoing in those follicles, but also the selective gene transcription of inhibin α and not CTBP. Taken together, our results suggest that CTBP mRNA expression in small follicles declines as the follicles develop and grow in response to FSH and hCG, suggesting a complex regulatory mechanism.

De novo Protein Synthesis is Required for the FSH-Induced Suppression of CTBP mRNA Expression.

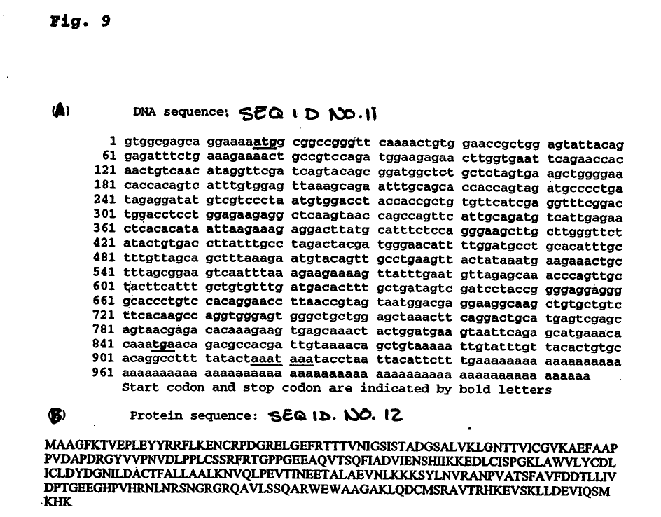

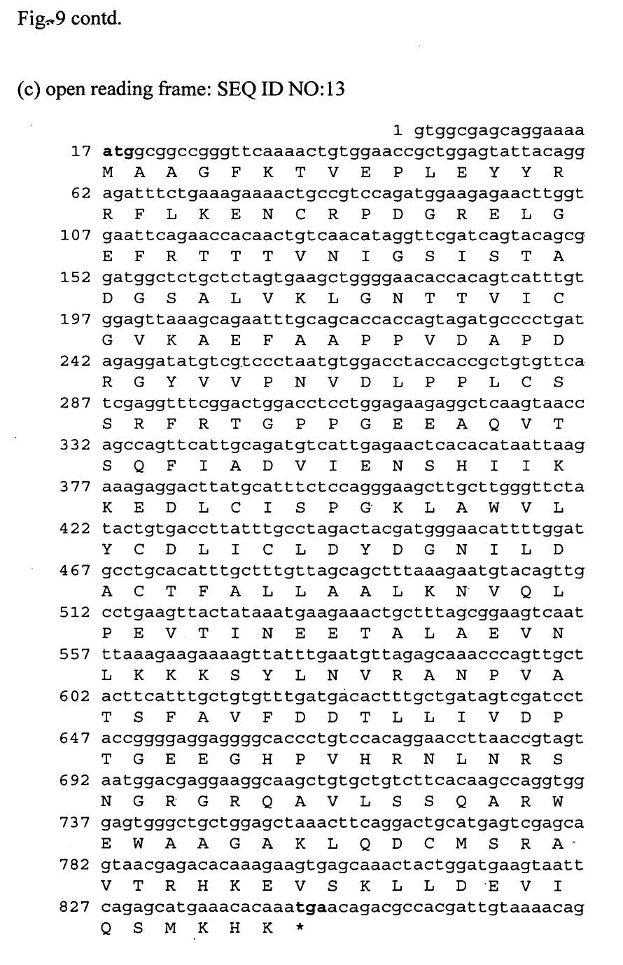

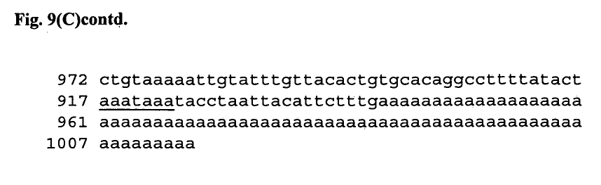

The FSH dependent CTBP mRNA decrease takes time since it was noticeable by six hours exposure to FSH, but not three hours exposure (FIGS. 4 and 5). This is in contrast to the quick response of the cytoskeletal gene expression within three hours of exposure to FSH (unpublished observation). This and the putative complex regulatory mechanism of the CTBR mRNA regulation raise the logical question whether or not FSH is directly involved in the decline. To address this question, the effects of cycloheximide (CHX), a translation inhibitor, and α-amanitin, a transcription inhibitor, were examined. Granulosa cells isolated from the ovaries of immature rats primed with 17β-estradiol were cultured for six hours with or without FSH and in the presence or absence of antibiotics. RNA was extracted and CTBP mRNA was measured by RT-PCR with the internal control, L-19. In the presence of CHX, the FSH-dependent decrease in CTBP mRNA was significantly less than in the absence of CHX (FIG. 6). The result demonstrates that de novo synthesis of a protein(s) is involved in the FSH-dependent decline of CTBP mRNA. In contrast, CHX treatment increased the CTBP mRNA level in the absence of FSH, suggesting the possibility that de novo protein synthesis is also involved in maintaining the steady state of CTBP mRNA in granulosa cells of the small follicles. It is possible that the protein is an RNase, since it is associated with the decrease in the CTBP mRNA level. The nucleotide sequence and amino acid sequence of the cDNA and its encoded protein are shown in FIG. 9.

Since, the FSH-dependent down regulation of CTBP mRNA involves de novo synthesis of a protein, we determined whether the protein synthesis involves transcriptional regulation. To this end, we tested the effect of α-amanitin, a transcription inhibitor. Co-treatment with FSH and α-amanitin completely abolished CTBP mRNA expression, whereas α-amanitin alone did not significantly impact the expression level as compared to the control (FIG. 6). A simple explanation of these results is that the de novo synthesis of the protein factor does not require transcription. It is unclear, however, whether the FSH-dependent down regulation of CTBP mRNA involves suppression of gene transcription, although the mRNA level of a gene is likely transcriptionally regulated.

FSH is capable of activating two distinct signal pathways, the adenylyl cyclase/cAMP pathway and phospholipase Cβ/inositol phosphate and diacyl glycerol pathway. We have previously demonstrated that FSH activates the adenylyl cyclase/cAMP pathway to quickly induce the massive reorganization of the cytoskeletons with dramatic morphological changes (Grieshaber et al., 2000. Endocrinology 141:3461-70). To determine whether FSH down-regulates CTBP mRNA via the adenylyl cyclase/cAMP, the cells were treated with forskolin, instead of FSH, which activates adenylyl cyclase and induces cAMP production. Forskolin simulated the effect of FSH (FIG. 6), as the drug treatment reduced the CTBP mRNA level, which was partially prevented by cycloheximide, thus confirming the FSH action.

Tissue Specific Expression of CTBP mRNA

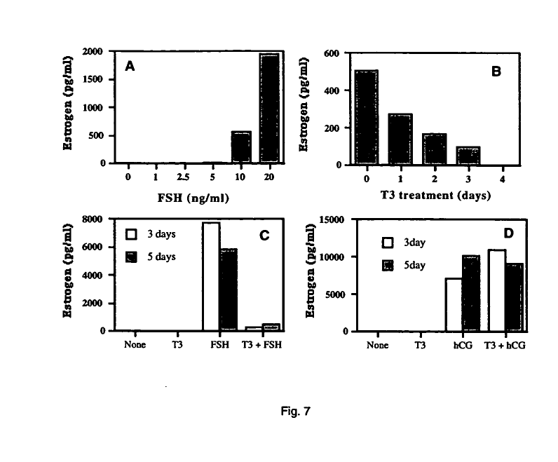

Because this is the first study on CTBP mRNA expression in the rat, we examined the tissue distribution of the CTBP transcript. Total RNA was isolated from the liver, stomach, pancreas, lung, bladder, kidney, intestine, brain and cerebellum of an adult female rat, the testis of an adult male rat, and ovaries of immature rats primed with PMSG with or without hCG. The mRNA appeared in a band of 1.3 kb (FIG. 7), suggesting a single transcript. Its expression could not be detected in the stomach, pancreas, lung, bladder, intestine and testis. The brain showed the highest level of the mRNA, but the cerebellum of the brain did not, suggesting site specific expression in the brain. Expression was abundant also in the liver and kidney. In addition to these tissues, the CTBP mRNA level in ovaries was examined to directly verify the RT-PCR and in situ hybridization results shown in FIGS. 3-6. The Northern blot results are consistent with all other observations. For example, the adult rat ovary showed a relatively low level of CTBP mRNA. However, the mRNA level was significant in the immature rat ovary primed with PMSG for 12 hours, but gradually declined as the rats were primed with PMSG for longer periods and additionally with hCG. These results verify that CTBP mRNA is responsive to FSH, and the hormone down-regulates the gene transcripts. In addition, the tissue specific expression suggests a role of CTBP in tissues other than the eye lens.

Effects of Thyroid Hormone T3 on estrogen production

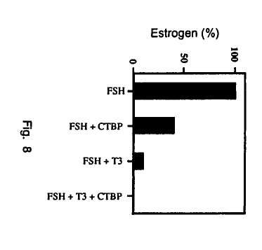

CTBP binds thyroid hormone, and therefore, we set out to determine the effect of thyroid hormone, T3, on granulosa cells. FSH induced estrogen production in a dose dependent manner (FIG. 8A) as expected, which was suppressed by T3 within 24 h and completely blocked by day 4 (FIG. 8B). T3 suppresses the FSH-induced estrogen synthesis (FIG. 8C). In contrast, T3 stimulates hCG-dependent estrogen production (FIG. 8D). These results show that T3 up- or down-regulates estrogen production dependent on hCG or FSH. Since CTBP carries T3 and should play a key role in the regulation of estrogen production.

These data demonstrate that CTBP mRNA is expressed in the ovary, particularly in the granulosa cell layer of preantral and early antral follicles, but not in large preovulatory follicles. Its expression is responsive to FSH, which is dependent on granulosa cell differentiation and follicular development. FSH down-regulates the mRNA via the adenylyl cyclase/cAMP/protein kinase A pathway, and mainly by a post-transcriptional mechanism. The down-regulation requires de novo synthesis of a regulatory protein(s) and the CTBP mRNA level is likely regulated by mRNA degradation.

In agreement with our Northern blot result (FIG. 7), a similar tissue specific expression pattern CTBP mRNA (FIG. 7) was observed in the mouse (Aoki et al., 2000 J Invest Dermatol 115:402-5). However, expression of CTBP transcript in the ovaries has never been described previously. However, the data reported herein demonstrate that CTBP mRNA is expressed in rat granulosa cells is demonstrated. This includes several lines of evidence: differential display, semi-quantitative RT-PCR, in situ hybridization and Northern blot. Moreover, we examined several different targets: ROG cells, primary granulosa cell culture and entire ovaries at various stages displaying follicles in a wide range of development. These rigorous examinations lead to a number of interesting and potentially significant observations. The conclusion that FSH impacts expression of CTBP mRNA is based on the following observations. FSH treatment for 6 h consistently resulted in the noticeable decline of the CTBP mRNA level in ROG cells, primary granulosa cell cultures and in growing follicles in the PMSG-primed rat ovaries. This FSH-responsive and preferential expression in preantral and early antral follicles suggests that the expression is dependent on granulosa cell differentiation and follicular development. Consistently, primordial follicles also expressed CTBP mRNA, but large antral follicles showed no or only marginal levels of the mRNA. The down-regulation of CTBP mRNA by FSH in granulosa cells was mediated, at least in part, by the adenylyl cyclase/cAMP signal pathway, because forskolin simulated the FSH action. The CTBP mRNA level appears to decline by mRNA degradation as well as transcription inhibition, but it is not clear how much of the transcriptional inhibition is responsible for the FSH-induced down regulation. On the other hand, the down regulation clearly requires de novo synthesis of a protein(s), likely from the existing mRNA.

Recently, it has been shown that adequate levels of circulating T₃ are important for normal female reproductive functions. Changes in T₃ levels result in menstrual disturbances, impaired fertility, and altered pituitary gonadotropin secretion in humans and animals. T₃ modulates FSH and LH action on steroidogenesis in porcine and human granulosa cells in vitro. Consistent with these observations, T₃ binding protein and T₃ receptor mRNA have been found in mammalian granulosa cells.

Some actions of T₃ are exerted by its direct contact to target molecules. However, T₃ is widely recognized for binding to nuclear receptors and regulating transcription. These T₃ receptors belong to the super family of ligand-dependent transcription factors that include the receptors for steroids, retinoids and vitamin D. The steroid receptors have four general functions, binding steroids, shuttling between the cytosol and nucleus, transporting the steroid, and interacting with genes in the nucleus to regulate transcription. In contrast, the T₃ receptors do not shuttle between the cytosol and nucleus, and therefore, cannot transport the ligand, T₃, from the cytosol to the nucleus. Instead, T₃ receptors remain bound to their target genes, regardless of ligand binding. Therefore, the thyroid receptors need a cytosolic ligand carrier to transport thyroid hormones from the cytosol to the nucleus. It will be interesting to see whether CTBP fulfills the role of the carrier.

It has been shown that T₃ not only directly inhibits the aromatase activity, but also down regulates the aromatase mRNA expression. Therefore, to enhance the aromatase activity and estrogen production, it is logical for FSH to reduce the T₃ level in granulosa cells. A simple way is to lower the level of the thyroid hormone carrier in granulosa cells as FSH down-regulated CTBP mRNA shown in this study, which would deny T₃ access to the sites of the aromatase activity and synthesis of aromatase mRNA. This would provide three approaches for FSH to induce aromatase by activation of the enzyme activity, abrogation of the suppression of the aromatase gene transcription and directly increasing in the aromatase gene transcription as generally established.

In conclusion, CTBP was identified as a FSH-responsive gene in granulosa cells. Messenger RNA encoding this protein is abundantly expressed in immature follicles, but upon exposure to FSH, the transcript level sharply decreased to an undetectable level. This down-regulation is accomplished via the adenylyl cyclase/cAMP/protein kinase A pathway, by de novo synthesis of a regulatory protein(s). This down-regulation of CTBP may be an integral part of the FSH-induced surge of estrogen production in granulosa cells.

Thus, this by modulating the T3 binding protein gene and its products it is possible to control estrogen production, other steroidogenesis, follicular development, ovulation cycles and pregnancy. Such modulation is accomplished in mammals by administering an effective amount of FSH to the mammal. An effective amount of FSH is that which is required to suppress expression of the T3 binding protein gene, as demonstrated by the example below.

The inventors isolated a nucleic acid molecule having the sequence of SEQ ID No.11 and showed that the expression of the gene encoded thereby (SEQ ID NO. 13) is modulated by FSH. In particular, the gene is up-regulated in the presence of FSH. The amino acid sequence of the protein encoded by SEQ ID NO. 12 is shown in FIG. 9 (SEQ ID NO. 13). Thus, FSH can be used to regulate the expression of this gene and other nucleic acid molecules that hybridize to SEQ ID NO.13 under high stringency conditions. Generally, high stringency conditions are used in the screening process. Methods for selection of stringency conditions are well known to those of skill in the art. See, e.g., Maniatis et al., Molecular Cloning A Laboratory Manual. An example of highly stringent wash conditions is 0.15 M NaCl at 72° C. for about 15 minutes. An example of stringent wash conditions is a 0.2×SSC wash at 65° C. for 15 minutes (See, Sambrook et al. (1989) Molecular Cloning—A Laboratory Manual (2^{nd} ed.) Vol. 1-3, Cold Spring Harbor Laboratory, Cold Spring Harbor Press, N.Y., for a description of SSC buffer and description of stringency conditions for nucleic acid hybridization). Often, a high stringency wash is preceded by a low stringency wash to remove background probe signal. An example of a medium stringency wash for a duplex (e.g., of more than 100 nucleotides), is 1×SSC at 45° C. for 15 minutes. An example of low stringency wash for a duplex (e.g., of more than 100 nucleotides), is 4-6×SSC at 40° C. for 15 minutes. In general, a signal to noise ratio of 2× (or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, for example, when a copy of a nucleic acid is created using the maximum codon degeneracy permitted by the genetic code.

### EXAMPLE 1

Materials

Dulbecco's modified Eagle's Media (DMEM), Hams-F12 and antibiotics for tissue culture were from Gibco-BRL (Gaithersburg, Md.). Restriction enzymes, reverse transcriptase, T7 and SP6 RNA polymerases, and Taq DNA polymerase were obtained from New England Biolabs (Beverly, Mass.). [α-³²S]UTP and [α-³²P]dCTP were from New Amersham Pharmacia Biotech (Piscataway, N.J.). Oligonucleotides were synthesized by Sigma. (Coralville, Iowa). FSH, hCG and activin A were purchased from the National Hormone and Peptide Program. Pregnant mare's serum gonadotropin (PMSG) was purchased from Sigma.

Animals, Hormone Treatment, Granulosa Cell Isolation and Culture

ROG cells were cultured as previously described by Li, et al. 1997 Endocrinology 138:2648-2657. Briefly, ROG cells were maintained in suspension in a defined serum free medium consisting of F12-Dulbecco's modified Eagle's medium (DMEM) supplemented with activin A (25 ng/ml), insulin (10 μg/ml), transferrin (5 μg/ml), α-tocopherol (0.1 μg/ml), progesterone (10 nM), bovine serum albumin (0.1%), and aprotinin (25 μg/ml) in the absence of antibiotics. Activin A (25 ng/ml) was replenished every 24 hours. The cells were provided with fresh media once a week, pooled every two weeks by centrifugation at 1000 rpm for 5 min and replated at 1:2.

All animals were handled according to the guidelines for care and use of animals set by the National Institutes of Health and the University of Kentucky Institutional Animal Care and Use Committee. Eighteen to twenty-one day old Sprague-Dawley female pups with nursing mothers were purchased from Harlan Breeding Company (Indianapolis, Ind.) and housed in a photoperiod of 14 h light/10 h darkness with light on at 0500 h. For in situ hybridization analysis, rats were injected s.c. with 15 IU PMSG in 0.1 ml PBS at 22 or 23 days of age. Some of the rats primed with PMSG for 48 h were additionally injected i.p. with 10 IU hCG.

For granulosa cell culture, immature rats were daily injected sub cutaneously with 1.5 mg of 17β-estradiol at 21, 22 and 23 days of age. Ovaries were isolated from the rats on day 24 and granulosa cells exhibiting a small antral phenotype were collected in cold serum-free 4F medium consisting of 15 mM HEPES (pH 7.4), 50% DMEM and 50% Ham's F12 with bovine transferrin (5 μg/ml), human insulin (2 mg/ml), hydrocortisone (40 ng/ml) and antibiotics. After cells were washed three times in 4F, they were plated on serum-coated, 6-well plates at a density of ˜1×10⁶ cells per cell and incubated in the humidified atmosphere of 5% CO₂ at 37 C. After 16 h, FSH (30 ng/ml) or forskolin (10 μM) was added to the cultures. For the inhibition of protein synthesis or transcription, cycloheximide (10 μg/ml) or α-amanitin (30 μg/ml) was added, respectively, 1 h before hormone treatment.

Differential Display

ROG cells were incubated in the absence of FSH (0 h) or presence of FSH (30 ng/ml) for 6 h in triplicate and total RNA was extracted. Pooled total RNA was used as a template for differential display of mRNA analyses using the Delta^{™} Differential Display Kit (Clonetech Laboratories, Inc., Palo Alto, Calif.) according to the manufacturer's instruction. cDNA fragments were re-amplified by PCR, cloned into PCR 2.1 TA cloning vector (Invitrogen), and sequenced on a Beckman CEQ 2000 capillary sequencer.

Northern Blot

For Northern analysis, 4-20 μg of total RNA per sample was resolved on 1.2% agarose gels containing 2.2 M formaldehyde and blotted to nylon membranes (Nytran super charge, Schleicher & Schuell Keene, NH). [α-³²P]dCTP-labeled cDNA probes were prepared from the CTBP clone using random primers. Blots were hybridized overnight at 42 C in 50% (v/v) formamide, 5×SSPE, 5× Denhardt's reagent, 0.1% (w/v) SDS, and 200 mg/ml denatured, fragmented herring testis DNA. Filters were washed once at low stringency (5×SSPE, 0.1% SDS, 25 C) and twice at high stringency (0.1×SSPE, 1% SDS, 62 C) for 45 minutes and visualized on phosphoimager (Fuji FLA-2000).

Reverse Transcription-Polymerase Chain Reaction (RT-PCR)

RT-PCR was performed as previously described (Ko et al. 1999 Endocrinology 140:5185-519411). Total RNA (1-2 μg) was reverse-transcribed at 37° C. in 20 μl using random hexamer (500 ng) and MMLV reverse transcriptase (10 units) (New England BioLabs, Boston, Mass.). Complementary DNA (cDNA) in 2 μl was added for a total 25 μl reaction mixture containing the primers (200 ng each), 0.4 mM dNTP mixture, and Taq DNA polymerase (2.5 U) in 1×PCR buffer (10 mM Tris, pH 8.3, 50 mM KCl, 1.5 mM MgCl₂, 0.01% gelatin). All PCR amplifications were carried out for 20, 25, 30 cycles on a MJ research Minicycler (MJ Research, MA). PCR products were separated by 2% agarose gel electrophoresis, stained with SYBR® Green I (Molecular Probes), and visualized on a phosphoimager. The primers were 5′-ctg act ggc gag aac tgg atg-3′SEQ ID NO. 1) and 5′-aca gta tgc agg ctt cgc tcc-3′ (SEQ ID NO. 2) for 160 bp CTBP, 5′-gct ttc cct ctg ttg acc cac-3′(SEQ ID NO. 3) and 5′-aga tgt tga ggg cag ctc gat-3′(SEQ ID NO. 4) for 255 bp inhibin α, 5′-ctg aag gtc aaa ggg aat gtg-3′ (SEQ ID NO. 5) and 5′-gga cag agt ctt gat gat ctc-3′ (SEQ ID NO. 6) for 194 bp L-19 as an internal control.

In situ Hybridization

Frozen ovaries were cut in 20 μm sections using a MICROM HM 505 E cryostat (Microm Labogerate GmbH, Germany) and mounted onto Superfrost/Plus Microscope slides (Fisher, Pa.). Sections were fixed, pre-treated and hybridized with antisense and sense RNA probes as previously described (Ko et al. Endocrinology 140:5185-519411). Using T7 or SP6 polymerase, [α-³⁵S]UTP-labeled RNA probes were synthesized from clones in pBluescript II vector (Stratagene). RNA probes (10⁷ cpm/ml) in hybridization buffer consisting of 50% formamide, 5×SSPE, 2× Denhardt's reagent, 10% dextran sulfate, 0.1% SDS and 100 μg/ml yeast tRNA were applied to sections, which were incubated in a humidity chamber at 47° C. for 16-18 hours. After hybridization, the sections were treated with RNAse A (20 μg/ml) at 37° C. for 30 min, washed repeatedly in increasingly lower concentrations of SSC down to 0.1×SSC at 58° C., and dehydrated through an ethanol series. The slides were exposed to Kodak BIOMAX MR film for 2 days and processed for liquid emulsion autoradiography using NTB-2 emulsion (Kodak, Rochester, N.Y.) for three to six weeks. Developed sections were stained with Gill's Formulation #2 hematoxylin solution (Fisher Scientific). Tissues were examined on a Nikon Microphot-SA microscope (Nikon, Melville, N.Y.) under bright- and dark field optics. Sense riboprobes were used as a control for nonspecific binding.

1. A method of modifying cytosolic T₃-binding protein (CTBP) gene expression comprising contacting the cytostolic T₃-binding protein gene with an effective amount of follicle stimulating hormone (FSH).

2. The method of claim 2 wherein the expression of CTBP is down regulated.

3. A method for modifying preantral stage and/or early antral stage follicular development in a mammal comprising exposing the follicles of the mammal to an effective amount of a compound that activates the adenylyl cyclase/cAMP signal pathway.

4. The method of claim 3 wherein the compound is FSH or forskolin.

5. The method of claim 3 wherein follicular development is suppressed.

6. A method of modifying CTBP gene expression in granulosa cells comprising contacting the granulosa cells with an effective amount FSH.

7. A method of enhancing aromatase activity in granulosa cells comprising contacting the granulosa cells with an amount of FSH effective to suppress CTBP gene expression.

8. A method of modifying estrogen production in a mammal comprising administering to the mammal an effective amount of follicle stimulating hormone.

9. A method of modifying ovulation in a mammal comprising administering to the mammal an effective amount of follicle stimulating hormone.

10. An isolated nucleic acid molecule having the nucleotide sequence of SEQ ID NO. 12 or having a nucleotide sequence that hybridizes under high stringency conditions to the complement of SEQ ID NO. 12 and comprises a regulatory region that is modulated by FSH.

11. The isolated nucleic acid molecule of claim 10 wherein the nucleic acid molecule is up-regulated in the presence of FSH.

12. An isolated polypeptide of SEQ ID NO. 13.

13. A method of modifying the expression of the polypeptide of SEQ ID NO. 13 comprising exposing the gene encoding the polypeptide to an effective amount of FSH.


SEQUENCE LISTING (13 sequences)

SEQ ID NO: 1 - DNA, 21 bp, Artificial Sequence
  Chemically synthesized
ctgactggcgagaactggatg

SEQ ID NO: 2 - DNA, 21 bp, Artificial Sequence
  Chemically synthesized
acagtatgcaggcttcgctcc

SEQ ID NO: 3 - DNA, 21 bp, Artificial Sequence
  Chemically synthesized
gctttccctctgttgacccac

SEQ ID NO: 4 - DNA, 21 bp, Artificial Sequence
  Chemically synthesized
agatgttgagggcagctcgat

SEQ ID NO: 5 - DNA, 21 bp, Artificial Sequence
  Chemically synthesized
ctgaaggtcaaagggaatgtg

SEQ ID NO: 6 - DNA, 21 bp, Artificial Sequence
  Chemically synthesized
ggacagagtcttgatgatctc

SEQ ID NO: 7 - DNA, 1099 bp, Rattus norvegicus
caggcggcgagatgaggcgggcgccagcgtttctgagcgccgacgaggtgcaggaccacctccgcagctccagcctcctcatcccgcccctggaggccgcactggccaacttctccaaaggtcccgacggaggggtcatgcaaccggtgcgcaccgtggtgcctgtggccaagcaccgaggcttcttgggagtcatgccagcctacagtgccgctgaggatgcactcaccaccaagttagtcaccttctatgagggccacagcaacaatgctgtcccctcccaccaggcatcagtgcttctctttgatcccagcaatggttccctgctggcggtcatggatggaaatgtcataactgcaaagaggacagcagccgtctctgccatcgccaccaagtttttgaagcccccaggcagtgatgtgctgtgcattcttggggctggggtccaggcgtacagtcactatgagatcttcacagaacagttctccttcaaggaggtgagaatgtggaaccgcaccagggaaaatgctgagaagtttgcaagctcagtgcagggagatgttcgggtctgttcatcagtgcaggaggctgtgacaggtgccgatgtcatcatcacagtcaccatggcaacggagcccattttatttggtgaatgggtgaagcccggggctcacatcaatgctgttggagccagtagacctgactggcgagaactggatgacgagctcatgaagcaagcagtgctgtatgtggactcccgggaggctgccctaaaggagtcaggagatgttctgttgtcaggggctgacatctttgctgagcttggagaagtggtttcaggagcgaagcctgcatactgtgagaagaccacggtgttcaagtctttggggatggcagtggaggacctggtcgcagccaaattagtgtacgattcgtggtcatctggcaagtgagcagaaggagctgtgcctgggctggatggacgtcacggctcaaacgctggctcagtgtctagatcaaaggaggcctagtccccagtgaacgggagtgagagtcactcataagtattgacatccctattcatgtttgtggttggata

SEQ ID NO: 8 - protein, 313 aa, Rattus norvegicus
MRRAPAFLSADEVQDHLRSSSLLIPPLEAALANFSKGPDGGVMQPVRTVVPVAKHRGFLGVMPAYSAAEDALTTKLVTFYEGHSNNAVPSHQASVLLFDPSNGSLLAVMDGNVITAKRTAAVSAIATKFLKPPGSDVLCILGAGVQAYSHYEIFTEQFSFKEVRMWNRTRENAEKFASSVQGDVRVCSSVQEAVTGADVIITVTMATEPILFGEWVKPGAHINAVGASRPDWRELDDELMKQAVLYVDSREAALKESGDVLLSGADIFAELGEVVSGAKPAYCEKTTVFKSLGMAVEDLVAAKLVYDSWSSGK

SEQ ID NO: 9 - protein, 313 aa, Mus musculus
MKRAPAFLSAEEVQDHLRSSSLLIPPLEAALANFSKGPDGGVMQPVRTVVPVAKHRGFLGVMPAYSAAEDALTTKLVTFYEGHSNTAVPSHQASVLLFDPSNGSLLAVMDGNVITAKRTAAVSAIATKLLKPPGSDVLCILGAGVQAYSHYEIFTEQFSFKEVRMWNRTRENAEKFASTVQGDVRVCSSVQEAVTGADVIITVTMATEPILFGEWVKPGAHINAVGASRPDWRELDDELMRQAVLYVDSREAALKESGDVLLSGADIFAELGEVISGAKPAHCEKTTVFKSLGMAVEDLVAAKLVYDSWSSGK

SEQ ID NO: 10 - protein, 314 aa, Homo sapiens
MSRVPAFLSAAEEEDHLRSSSLLIPPLETALANFSSGEDGGVMQPVRTVVPVTKHRGYLGVMPAYSAAEDALTTKLVTFYEDRGITSVVPSHQATVLLFEPSNGTLLAVMDGNVITAKRTAAVSAIATKFLKPPSSEVLCILGAGVQAYSHYEIFTEQFSFKEVRIWNRTKENAEKFADTVQGEVRVCSSVQEAVAGADVIITVTLATEPILFGEWVKPGAHINAVGASRPDWRELDDELMEQAVLYVDSQEAALKESGDVLLSGAEIFAELGEVIKGVKPAHCEKTTVFKSLGMAVEDTVAAKLIYDSWSSGK

SEQ ID NO: 11 - DNA, 1015 bp, Rattus norvegicus
gtggcgagcaggaaaaatggcggccgggttcaaaactgtggaaccgctggagtattacaggagatttctgaaagaaaactgccgtccagatggaagagaacttggtgaattcagaaccacaactgtcaacataggttcgatcagtacagcggatggctctgctctagtgaagctggggaacaccacagtcatttgtggagttaaagcagaatttgcagcaccaccagtagatgcccctgatagaggatatgtcgtccctaatgtggacctaccaccgctgtgttcatcgaggtttcggactggacctcctggagaagaggctcaagtaaccagccagttcattgcagatgtcattgagaactcacacataattaagaaagaggacttatgcatttctccagggaagcttgcttgggttctatactgtgaccttatttgcctagactacgatgggaacattttggatgcctgcacatttgctttgttagcagctttaaagaatgtacagttgcctgaagttactataaatgaagaaactgctttagcggaagtcaatttaaagaagaaaagttatttgaatgttagagcaaacccagttgctacttcatttgctgtgtttgatgacactttgctgatagtcgatcctaccggggaggaggggcaccctgtccacaggaaccttaaccgtagtaatggacgaggaaggcaagctgtgctgtcttcacaagccaggtgggagtgggctgctggagctaaacttcaggactgcatgagtcgagcagtaacgagacacaaagaagtgagcaaactactggatgaagtaattcagagcatgaaacacaaatgaacagacgccacgattgtaaaacagctgtaaaaattgtatttgttacactgtgcacaggccttttatactaaataaatacctaattacattctttgaaaaaaaaaaaaaaaaaaaaaaaaaaaaaaaaaaaaaaaaaaaaaaaaaaaaaaaaaaaaaaaaaaaaaaaaa

SEQ ID NO: 12 - protein, 276 aa, Rattus norvegicus
MAAGFKTVEPLEYYRRFLKENCRPDGRELGEFRTTTVNIGSISTADGSALVKLGNTTVICGVKAEFAAPPVDAPDRGYVVPNVDLPPLCSSRFRTGPPGEEAQVTSQFIADVIENSHIIKKEDLCISPGKLAWVLYCDLICLDYDGNILDACTFALLAALKNVQLPEVTINEETALAEVNLKKKSYLNVRANPVATSFAVFDDTLLIVDPTGEEGHPVHRNLNRSNGRGRQAVLSSQARWEWAAGAKLQDCMSRAVTRHKEVSKLLDEVIQSMKHK

SEQ ID NO: 13 - DNA, 828 bp, Rattus norvegicus
atggcggccgggttcaaaactgtggaaccgctggagtattacaggagatttctgaaagaaaactgccgtccagatggaagagaacttggtgaattcagaaccacaactgtcaacataggttcgatcagtacagcggatggctctgctctagtgaagctggggaacaccacagtcatttgtggagttaaagcagaatttgcagcaccaccagtagatgcccctgatagaggatatgtcgtccctaatgtggacctaccaccgctgtgttcatcgaggtttcggactggacctcctggagaagaggctcaagtaaccagccagttcattgcagatgtcattgagaactcacacataattaagaaagaggacttatgcatttctccagggaagcttgcttgggttctatactgtgaccttatttgcctagactacgatgggaacattttggatgcctgcacatttgctttgttagcagctttaaagaatgtacagttgcctgaagttactataaatgaagaaactgctttagcggaagtcaatttaaagaagaaaagttatttgaatgttagagcaaacccagttgctacttcatttgctgtgtttgatgacactttgctgatagtcgatcctaccggggaggaggggcaccctgtccacaggaaccttaaccgtagtaatggacgaggaaggcaagctgtgctgtcttcacaagccaggtgggagtgggctgctggagctaaacttcaggactgcatgagtcgagcagtaacgagacacaaagaagtgagcaaactactggatgaagtaattcagagcatgaaacacaaa